(19) United States
(12) Patent Application Publication
(10) Pub. No.: US 2023/0255973 A1
(43) Pub. Date: Aug. 17, 2023

(54) COMBINATION THERAPY WITH A MUTANT IDH1 INHIBITOR, A DEOXYADENOSINE ANALOG, AND A PLATINUM AGENT

(71) Applicant: Eli Lilly and Company, Indianapolis, IN (US)

(72) Inventor: David Michael HYMAN, Westport, CT (US)

(21) Appl. No.: 18/005,300

(22) PCT Filed: Jul. 20, 2021
(86) PCT No.: PCT/US2021/042268

§ 371 Date: Jan. 12, 2023

Related U.S. Application Data
(60) Provisional application No. 63/053,895, filed on Jul. 20, 2020.

(51) Int. Cl. A61K 31/5365; A61K 31/7068; A61K 33/243; A61K 31/282; A61P 1/16; A61P 35/00

(52) U.S. Cl. CPC A61K 31/5365; A61K 31/7068; A61K 33/243; A61K 31/282; A61P 1/16; A61P 35/00

(57) ABSTRACT

The present invention relates to combination therapy with (a) a mutant IDH1 inhibitor, or a pharmaceutically acceptable salt thereof, (b) a deoxyadenosine analog, or a pharmaceutically acceptable salt thereof, and (c) a platinum agent, for the treatment of a solid tumor cancer.

The present invention relates to combination therapy with a mutant isocitrate dehydrogenase 1 (IDH1) inhibitor, or a pharmaceutically acceptable salt thereof, a deoxyadenosine analog, or a pharmaceutically acceptable salt thereof, and a platinum agent, for the treatment of a solid tumor cancer.

IDH1 is an enzyme that catalyzes the conversion of isocitrate to α-ketoglutarate (2-oxoglutarate), and reduces nicotinamide adenine dinucleotide phosphate (NADP⁺) to NADPH (Megias-Vericat J, et al., Blood Lymph. Cancer: Targets and Therapy 2019; 9: 19-32).

Mutations in IDH1, e.g., at IDH1 amino acid residue R132, contribute to tumorigenesis in several types of cancer, including solid tumors (Badur M G, et al., Cell Reports 2018; 25: 1680). IDH1 mutations can result in high levels of 2-hydroxyglutarate (2-HG), which inhibits cellular differentiation, and inhibitors of mutant IDH1 can reduce 2-HG levels, which promotes cellular differentiation (Molenaar R J, et al., Oncogene 2018; 37: 1949-1960). Mutations in IDH2, e.g., at IDH2 amino acid residues R140 and R172, also contribute to tumorigenesis (Kotredes K P, et al., Oncotarget 2019; 10: 2675-2692).

Certain mutant IDH1/IDH2 inhibitors are disclosed in WO 2018/111707 A1, including a compound defined herein as “Compound A,” which is a covalent inhibitor of mutant IDH1 that modifies a single cysteine (Cys269) in an allosteric binding pocket, rapidly inactivates the enzyme, and selectively inhibits 2-HG production, without affecting alpha-ketoglutarate a-KG levels (WO 2018/111707 A1).

Effective therapies for the treatment of solid tumor cancer, including cholangiocarcinoma, remain elusive.

In addition, so called “secondary” IDH1 mutations, as defined herein, may contribute to relapse after treatment with a mutant IDH1 inhibitor. For example, to date, six post-ivosidenib treatment secondary IDH1 mutations have been reported: R119P, G131A, D279N, S280F, G289D or H315D (Choe S, et al., “Molecular mechanisms mediating relates following ivosidenib monotherapy in subjects with IDH1-mutant relapsed or refractory acute myeloid leukemia,” 61^{st} Am. Soc. Hematol. (ASH) Annual Meeting poster, Dec. 7-10, 2019, Orlando, Fla., USA).

Thus, there exists a need for alternative treatments for solid tumor cancers, such as novel combination therapies.

The present invention provides a method of treating a solid tumor cancer, comprising administering to a subject having an IDH mutation a therapeutically effective amount of

(a) a compound of Formula I:

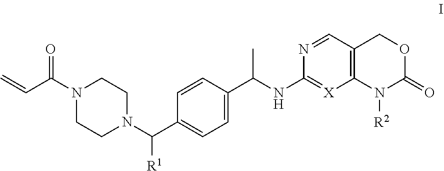

wherein:

R¹ is —CH₂CH(CH₃)₂, —CH₂CH₃, —CH₂CH₂OCH₃, or —CH₂-cyclopropyl;

R² is —CH₃ or —CH₂CH₃; and

X is N or CH;

or a pharmaceutically acceptable salt thereof; (b) a deoxyadenosine analog, or a pharmaceutically acceptable salt thereof; and (c) a platinum agent.

The present invention also provides a compound of Formula I:

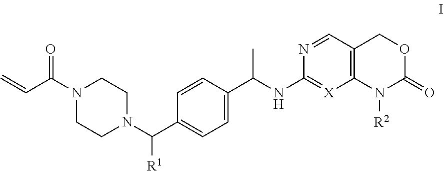

wherein:

R¹ is —CH₂CH(CH₃)₂, —CH₂CH₃, —CH₂CH₂OCH₃, or —CH₂-cyclopropyl;

R² is —CH₃ or —CH₂CH₃; and

X is N or CH;

or a pharmaceutically acceptable salt thereof;

for use in simultaneous, separate or sequential combination with gemcitabine, or a pharmaceutically acceptable salt thereof, and cisplatin, in the treatment of a solid tumor cancer in a subject having an IDH mutation. In another embodiment, the subject has been identified as having an IDH1 R132 mutation.

In one embodiment, the IDH mutation is an IDH1 mutation or an IDH2 mutation. In another embodiment, the IDH mutation is an IDH1 mutation. In another embodiment, the IDH1 mutation is an IDH1 R132 mutation. In another embodiment, the IDH1 mutation is R132H. In another embodiment, the IDH1 mutation is R132C, R132G, R132L, or R132S. In another embodiment, the IDH1 R132 mutation is R132H. In another embodiment, the IDH1 mutation is R132C. In another embodiment, the IDH1 mutation is R132G. In another embodiment, the IDH1 mutation is R132L. In another embodiment, the IDH1 mutation is R132S.

In another embodiment, the IDH mutation is an IDH2 mutation. In another embodiment, the IDH2 mutation is an IDH2 R140 mutation or an IDH2 R172 mutation. In another embodiment, the IDH2 mutation is an R140 mutation. In another embodiment, the R140 mutation is R140Q, R140L, or R140W. In another embodiment, the IDH2 mutation is an R172 mutation. In another embodiment, the R172 mutation is R172K, R172M, R172G, R172S or R172W.

In another embodiment of the method of the invention, the subject's solid tumor cancer has progressed after treatment with an IDH1 inhibitor compound other than the compound of Formula I. In another embodiment, the subject is intolerant to or is resistant to an IDH inhibitor other than the compound of Formula I. In another embodiment, the IDH inhibitor other than the compound of Formula I is ivosidenib or enasidenib. In another embodiment, the IDH inhibitor other than the compound of Formula I is ivosidenib. In another embodiment, the IDH inhibitor other than the compound of Formula I is enasidenib.

In one embodiment, X is N, or a pharmaceutically acceptable salt thereof. In another embodiment, X is N, R¹ is —CH₂-cyclopropyl, and R2 is —CH₂CH₃, or a pharmaceutically acceptable salt thereof. In another embodiment, X is N, le is —CH₂-cyclopropyl, and R2 is —CH₂CH₃.

In another embodiment, the compound of Formula I is:
7-[[(1S)-1-[4-[(1R)-2-cyclopropyl-1-(4-prop-2-enoylpiperazin-1-yl)ethyl]phenyl]ethyl]amino]-1-ethyl-4H-pyrimido[4,5-d][1,3]oxazin-2-one;
7-[[(1S)-1-[4-[(1S)-2-cyclopropyl-1-(4-prop-2-enoylpiperazin-1-yl)ethyl]phenyl]ethyl]amino]-1-ethyl-4H-pyrimido[4,5-d][1,3]oxazin-2-one; or
1-Ethyl-7-[[(1S)-1-[4-[1-(4-prop-2-enoylpiperazin-1-yl)propyl]phenyl]ethyl]amino]-4H-pyrimido[4,5-d][1,3]oxazin-2-one;
   or a pharmaceutically acceptable salt thereof.

In another embodiment, the compound of Formula I is 7-[[(1S)-1-[4-[(1S)-2-cyclopropyl-1-(4-prop-2-enoylpiperazin-1-yl)ethyl]phenyl]ethyl]amino]-1-ethyl-4H-pyrimido[4,5-d][1,3]oxazin-2-one.

In another embodiment, the compound of Formula I is:

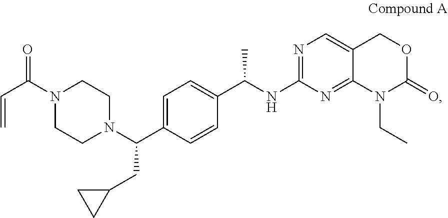 [SMILES: C=CC(=O)N1CCN([C@@H](CC2CC2)c2ccc([C@H](C)Nc3ncc4c(n3)N(CC)C(=O)OC4)cc2)CC1]

or a pharmaceutically acceptable salt thereof. In another embodiment, the compound of Formula I is Compound A.

In another embodiment, the deoxyadenosine analog is cytarabine or gemcitabine, or a pharmaceutically acceptable salt thereof. In another embodiment, the deoxyadenosine analog is gemcitabine, or a pharmaceutically acceptable salt thereof. In another embodiment, the deoxyadenosine analog is gemcitabine.

In another embodiment, the platinum agent is cisplatin, carboplatin or oxaliplatin. In another embodiment, the platinum agent is cisplatin.

In another embodiment, the compound of Formula I is:

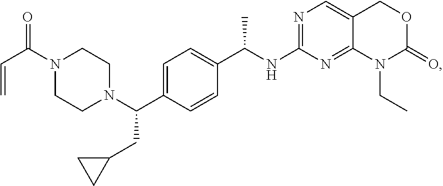 [SMILES: C=CC(=O)N1CCN([C@@H](CC2CC2)c2ccc([C@H](C)Nc3ncc4c(n3)N(CC)C(=O)OC4)cc2)CC1]

the deoxyadenosine analog is gemcitabine, and the platinum agent is cisplatin.

In another embodiment, the solid tumor cancer is cholangiocarcinoma, the compound of Formula I is

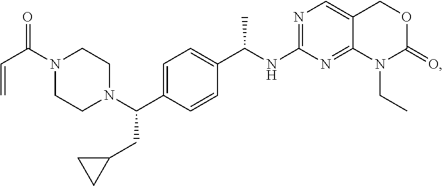 [SMILES: C=CC(=O)N1CCN([C@@H](CC2CC2)c2ccc([C@H](C)Nc3ncc4c(n3)N(CC)C(=O)OC4)cc2)CC1]

the deoxyadenosine analog is gemcitabine, and the platinum agent is cisplatin. In another embodiment, the cholangiocarcinoma is advanced cholangiocarcinoma.

In another embodiment, the compound of Formula I (e.g., Compound A) is administered at a dose of about 25, 50, 75, 100, 125, 150, 175, 200, 250, 300, 400, 600 or 800 mg once a day on each of days 1-21 of a 21 day cycle. In another embodiment, the compound of Formula I (e.g., Compound A) is administered at a dose of about 25, 50, 75, 100, 125, 150, 175, 200, 250 or 300 mg once a day on each of days 1-21 of a 21 day cycle. In another embodiment, the compound of Formula I (e.g., Compound A) is administered at a dose of about 25 mg on each of days 1-21 of a 21 day cycle. In another embodiment, the compound of Formula I (e.g., Compound A) is administered at a dose of about 50 mg on each of days 1-21 of a 21 day cycle. In another embodiment, the compound of Formula I (e.g., Compound A) is administered at a dose of about 75 mg on each of days 1-21 of a 21 day cycle. In another embodiment, the compound of Formula I (e.g., Compound A) is administered at a dose of about 100 mg on each of days 1-21 of a 21 day cycle. In another embodiment, the compound of Formula I (e.g., Compound A) is administered at a dose of about 125 mg on each of days 1-21 of a 21 day cycle. In another embodiment, the compound of Formula I (e.g., Compound A) is administered at a dose of about 150 mg on each of days 1-21 of a 21 day cycle. In another embodiment, the compound of Formula I (e.g., Compound A) is administered at a dose of about 175 mg on each of days 1-21 of a 21 day cycle. In another embodiment, the compound of Formula I (e.g., Compound A) is administered at a dose of about 200 mg on each of days 1-21 of a 21 day cycle. In another embodiment, the compound of Formula I (e.g., Compound A) is administered at a dose of about 250 mg on each of days 1-21 of a 21 day cycle. In another embodiment, the compound of Formula I (e.g., Compound A) is administered at a dose of about 300 mg on each of days 1-21 of a 21 day cycle. In another embodiment, the compound of Formula I (e.g., Compound A) is administered at a dose of about 400 mg on each of days 1-21 of a 21 day cycle. In another embodiment, the compound of Formula I (e.g., Compound A) is administered at a dose of about 600 mg on each of days 1-21 of a 21 day cycle. In another embodiment, the compound of Formula I (e.g., Compound A) is administered at a dose of about 800 mg on each of days 1-21 of a 21 day cycle.

In another, the compound of Formula I (e.g., Compound A) is administered at a dose of about 25, 50, 75, 100, 125, 150, 175, 200, 250 300, 400, 600 or 800 mg twice a day on each of days 1-21 of a 21 day cycle. In another, the compound of Formula I (e.g., Compound A) is administered at a dose of about 25, 50, 75, 100, 125, 150, 175, 200, 250 or 300 mg twice a day on each of days 1-21 of a 21 day cycle.

In another embodiment, gemcitabine is administered to the subject at a dose of about 1000 mg/m² on each of days 1 and 8 of a 21 day cycle.

In another embodiment, cisplatin is administered to the subject at a dose of about 25 mg/m² on each of days 1 and 8 of a 21 day cycle.

In another embodiment, gemcitabine is administered to the subject at a dose of about 1000 mg/m² on each of days 1 and 8 of a 21 day cycle, cisplatin is administered to the subject at a dose of about 25 mg/m² on each of days 1 and 8 of the 21 day cycle.

In another embodiment, a compound of Formula I (e.g., Compound A) is administered on each of days 1-21 of a 21 day cycle, gemcitabine is administered to the subject at a dose of about 1000 mg/m² on each of days 1 and 8 of the 21 day cycle, and cisplatin is administered to the subject at a dose of about 25 mg/m² on each of days 1 and 8 of the 21 day cycle.

In one embodiment, when the compound of Formula I (e.g., Compound A), gemcitabine, and cisplatin are administered on the same day, they are administered in the following order: the compound of Formula I, gemcitabine, and cisplatin.

In another embodiment, when the compound of Formula I (e.g., Compound A), gemcitabine, and cisplatin are administered on the same day, they are administered in the following order: the compound of Formula I, cisplatin, and gemcitabine.

In another embodiment, when the compound of Formula I (e.g., Compound A), gemcitabine, and cisplatin are administered on the same day, they are administered in the following order: compound of gemcitabine, the compound of Formula I, and cisplatin.

In another embodiment, when the compound of Formula I (e.g., Compound A), gemcitabine, and cisplatin are administered on the same day, they are administered in the following order: compound of gemcitabine, cisplatin, and the compound of Formula I.

In another embodiment, when the compound of Formula I (e.g., Compound A), gemcitabine, and cisplatin are administered on the same day, they are administered in the following order: compound of cisplatin, gemcitabine, and the compound of Formula I.

In another embodiment, when the compound of Formula I (e.g., Compound A), gemcitabine, and cisplatin are administered on the same day, they are administered in the following order: compound of cisplatin, the compound of Formula I (e.g., Compound A), and gemcitabine.

In another embodiment of the method of the invention, an antiemetic agent is administered to the subject prior to administration of gemcitabine and/or cisplatin.

In another embodiment, the subject is identified as having an IDH mutation. In another embodiment, the subject is identified as having one or more IDH1 mutations or one or more IDH2 mutations. In another embodiment, the subject is identified as one or more IDH1 mutations and one or more IDH2 mutations.

In another embodiment, the subject is identified as having an IDH mutation in solid tumor tissue. In another embodiment, the subject is identified as having an IDH1 mutation in solid tumor tissue cells. In another embodiment, the subject is identified as having an IDH1 mutation in peripheral blood.

In another embodiment of the method of the invention, the solid tumor cancer is cholangiocarcinoma, head & neck cancer, chondrosarcoma, hepatocellular carcinoma, melanoma, pancreatic cancer, astrocytoma, oligodendroglioma, glioma, glioblastoma, bladder carcinoma, colorectal cancer, or lung cancer. In another embodiment, the lung cancer is non-small cell lung cancer. In another embodiment, the lung cancer is non-small cell lung cancer, and a KRas G12C inhibitor and or an EGFR inhibitor is also administered. In another embodiment, the solid tumor is cholangiocarcinoma. In another embodiment, the cholangiocarcimona is advanced cholangiocarcinoma. In another embodiment, radiation therapy is also administered to the subject.

The present invention also provides the use of a compound of Formula I, or a pharmaceutically acceptable salt thereof, in the manufacture of a medicament for the treatment of a solid tumor cancer in a subject, wherein the medicament is administered in simultaneous, separate or sequential combination with gemcitabine, or a pharmaceutically acceptable salt thereof, and cisplatin.

In one embodiment, the solid tumor cancer is frontline cancer. In another embodiment, solid tumor the cancer is relapsed cancer. In another embodiment, the solid tumor cancer is refractory solid tumor cancer. In another embodiment, the solid tumor cancer is advanced solid tumor cancer. In another embodiment, the advanced solid tumor cancer is advanced cholangiocarcinoma.

In another embodiment, the subject has advanced solid tumor disease, and has not received prior therapy for advanced solid tumor disease. In another embodiment, the subject has advanced cholangiocarcinoma, and has not received prior therapy for advanced cholangiocarcinoma.

In another embodiment, the subject has one or more secondary IDH1 mutations. In another embodiment, the subject is identified as having one or more secondary IDH1 mutations.

As used above, and throughout the description of the invention, the following terms, unless otherwise indicated, shall be understood to have the following meanings:

The term “solid tumor issue” refers to tissue that is not hematologic tissue (hematologic tissue is blood, bone marrow, or lymphatic tissue). Non-limiting examples of solid tissue are cholangial tissue, pancreatic tissue, head tissue, neck tissue, hepatic tissue, skin tissue, astrocytomal tissue, oligodendroglial tissue, glial tissue, brain tissue, bladder tissue, colorectal tissue, and lung tissue.

The term “frontline solid tumor cancer” means that the solid tumor cancer subject has never been treated for the solid tumor cancer being treated.

The term “refractory solid tumor cancer” refers to cancer that has been treated, but the solid tumor cancer subject did not respond to treatment.

The term “relapsed solid tumor cancer” means that the solid tumor cancer subject responded to treatment for a period of time, but that the solid tumor cancer has reoccurred.

The term “advanced solid tumor cancer” refers to solid tumor cancer that has spread to lymph nodes or to other tissues outside of the solid tumor cancer's point of origin.

The term “cancer subject” means a subject who has been diagnosed with cancer.

The term “solid tumor subject” means a subject who has been diagnosed with a solid tumor cancer. In one embodiment, the solid tumor cancer is cholangiocarcinoma.

The term “IDH1 R132 mutation” refers to an IDH1 mutation at amino acid residue 132 in a subject's IDH1 gene, as determined, e.g., in the subject's nucleic acid (e.g., DNA).

The term “IDH2 R140 mutation” refers to an IDH2 mutation at amino acid residue 140 in a subject's IDH2 gene, as determined, e.g., in the subject's nucleic acid (e.g., DNA).

The term “IDH2 R172 mutation” refers to an IDH2 mutation at amino acid residue 172 in a subject's IDH2 gene, as determined, e.g., in the subject's nucleic acid (e.g., DNA).

The term “mutant IDH1 inhibitor” refers to a compound that inhibits the enzyme activity of and/or the production of 2-HG by a mutant IDH1 enzyme. Methods for assaying mutant IDH1 and IDH2 enzyme activity are known to those of ordinary skill in the art, e.g., in WO 2018/111707 A1.

The term “secondary IDH1 mutation” refers to an IDH1 mutation that occurs the IDH1 enzyme in a subject after treatment with a mutant IDH1 inhibitor other than a compound of Formula I herein. In one embodiment, the one or more secondary IDH1 mutations is one or more of R119P, G131A, D279N, S280F, G289D or H315D in IDH1. However, other secondary IDH1 mutations may be reported in the future. As used herein, a “secondary IDH1 mutation” is not an “IDH1 R132 mutation,” an “IDH2 R140 mutation,” or an “IDH2 R172 mutation.”

The term “identified as having an IDH1 R132 mutation” means that nucleic acid (e.g., DNA) from the subject's tissue or cells (e.g., circulating tumor cells) has been analyzed to determine if a subject has an IDH1 R132 mutation. In one embodiment, the subject's blood cells, bone marrow cells, or blood cells and bone marrow has been analyzed for an IDH1 R132 mutation. In another embodiment, the subject's solid tissue has been analyzed for an IDH1 R132 mutation.

The term “identified as having an IDH2 R140 mutation” means that nucleic acid (e.g., DNA) from the subject's tissue or cells has been analyzed to determine if a subject has an IDH2 R140 mutation. In one embodiment, the subject's blood cells, bone marrow cells, or blood cells and bone marrow has been analyzed for an IDH1 R140 mutation. In another embodiment, the subject's solid tissue has been analyzed for an IDH1 R140 mutation.

The term “identified as having an IDH2 R172 mutation” means that nucleic acid (e.g., DNA) from the subject's tissue or cells has been analyzed to determine if a subject has an IDH2 R172 mutation. In one embodiment, the subject's blood cells, bone marrow cells, or blood cells and bone marrow has been analyzed for an IDH2 R172 mutation. In another embodiment, the subject's solid tissue has been analyzed for an IDH2 R172 mutation.

In one embodiment, the party who identifies the subject as having an IDH mutation (e.g., one or more of an IDH1 R132 mutation, IDH2 R140 mutation or IDH2 R172 mutation) is different than the party that administers a compound of formula I, or a pharmaceutically acceptable salt thereof, a deoxyadenosine analog, or a pharmaceutically acceptable salt thereof, and a platinum agent. In another embodiment, the party who identifies the subject as having an IDH mutation (e.g., one or more of an IDH1 R132 mutation, IDH2 R140 mutation or IDH2 R172 mutation) is the same as the party that administers a compound of formula I, or a pharmaceutically acceptable salt thereof, a deoxyadenosine analog, or a pharmaceutically acceptable salt thereof, and a platinum agent.

Analytical methods for identifying IDH mutations are known to those of ordinary skill in the art (Clark, O., et al., Clin. Cancer. Res. 2016; 22: 1837-42), including, but not limited to, karyotyping (Guller J L, et al., J. Mol. Diagn. 2010; 12: 3-16), fluorescence in situ hybridization (Yeung D T, et al., Pathology 2011; 43: 566-579), Sanger sequencing (Lutha, R et al., Haematologica 2014; 99: 465-473), metabolic profiling (Miyata S, et al., Scientific Reports 2019; 9: 9787), polymerase chain reaction (Ziai, J M and A J Siddon, Am. J. Clin. Pathol 2015; 144: 539-554), and next-generation sequencing (e.g., whole transcriptome sequencing) (Lutha, R et al., Haematologica 2014; 99: 465-473; Wang H Y, et al., J. Exp. Clin. Cancer Res. 2016; 35: 86.

The term “about” means±5% of the numerical value recited.

The terms “treatment,” “treat,” “treating,” and the like, are meant to include slowing, stopping, or reversing the progression of cancer. These terms also include alleviating, ameliorating, attenuating, eliminating, or reducing one or more symptoms of a disorder or condition, even if the cancer is not actually eliminated and even if progression of the cancer is not itself slowed, stopped or reversed.

“Therapeutically effective amount” means the amount of a compound, or pharmaceutically acceptable salt thereof, administered to the subject that will elicit the biological or medical response of or desired therapeutic effect on a subject. A therapeutically effective amount can be readily determined by the attending clinician, as one skilled in the art, by the use of known techniques and by observing results obtained under analogous circumstances. In determining the effective amount for a subject, a number of factors are considered by the attending clinician, including, but not limited to: size, age, and general health; the specific disease or disorder involved; the degree of or involvement or the severity of the disease or disorder; the response of the individual subject; the particular compound administered; the mode of administration; the bioavailability characteristics of the preparation administered; the dose regimen selected; the use of concomitant medication; and other relevant circumstances.

A “pharmaceutically acceptable carrier, diluent, or excipient” is a medium generally accepted in the art for the delivery of biologically active agents to mammals, e.g., humans.

The compounds administered according to the invention can optionally be formulated as pharmaceutical compositions administered by any route which makes the compound bioavailable. In an embodiment, such compositions are formulated for oral administration. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006).

“Pharmaceutically acceptable salts” or “a pharmaceutically acceptable salt” refers to the relatively non-toxic, inorganic and organic salt or salts of the compound of the present invention (S. M. Berge, et al., “Pharmaceutical Salts”, Journal of Pharmaceutical Sciences, Vol 66, No. 1, January 1977).

It will be understood by one of ordinary skill in the art that compounds administered according to the invention are capable of forming salts. The compounds react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008).

### EXAMPLE 1

### In Vivo Tumor Growth Inhibition in IDH1 Mutant Cholangiocarcinoma PDX Tumor Model

Compounds and Formulation. For in vivo studies, each test article is prepared at an appropriate concentration with vehicle. Compound A is formulated in Acacia vehicle (water, 10% Acacia, 0.05% Antifoam [Dow Corning 1510-US]) with 1.1 molar equivalents HCl. Compound A is prepared fresh at the appropriate concentration with vehicle every 7 days and stored at 4° C. between doses. Stock Cisplatin injection solution (Teva Pharmaceuticals, NDC #00703-5747-11) is stored at room temperature. On the day of administration, the appropriate concentration is prepared by dilution in 0.9% sterile saline. Gemcitabine is prepared fresh weekly in 0.9% sterile saline.

In Vivo Tumor Growth Inhibition Study in IDH1 Mutant (R132C) Intrahepatic Cholangiocarcinoma PDX Tumor Model. Tumor fragments are harvested from host animals and subcutaneously implanted into 6-12 week old immune-deficient female mice. Mice are fed ad libitum on normal chow. The study is initiated at a mean tumor volume of approximately 125-250 mm³. Each test article is prepared as described above at the appropriate concentration with vehicle to give animals the doses tested in this study at a dosing volume of 10 μL/gram body weight. Mice are administered Compound A (30 mg/kg, PO, QD) on day 0 by oral gavage and are treated for the duration of the study (day 31). On day 5 of Compound A dosing and Q7D×3 thereafter, prepare gemcitabine and cisplatin in appropriate vehicle. Dose gemcitabine (40 mg/kg) and cisplatin (1.5 mg/kg) by intraperitoneal administration. Tumor growth and body weight are monitored over time to evaluate efficacy and signs of toxicity. Bidimensional measurements of tumors are performed twice a week and tumor volumes are calculated based on the following formula: (Tumor Volume)=[(L)×(W²)×0.52] where L is mid-axis length and W is mid-axis width. Mean tumor volumes on day 31 are shown in Table 1.

Compound A, cisplatin plus gemcitabine, and the triple combination of Compound A with cisplatin and gemcitabine are found to have delta T/C % values as provided in Table 1 below. These results indicate that the combination of Compound A with cisplatin and gemcitabine results in a statistically significant additive benefit to tumor growth inhibition in a mutant IDH1 (R132C) cholangiocarcinoma patient-derived xenograft model. The addition of Compound A to the cisplatin-gemcitabine regimen demonstrates no evidence of antagonism or overt toxicity, as compared to the cisplatin-gemcitabine treatment alone.

TABLE 1
In vivo tumor growth inhibition in IDH1 mutant cholangiocarcinoma
PDX tumor model implanted in mice

| Group | N | Mean ± SEM | Delta T/C % | n-value |
|---|---|---|---|---|
| Vehicle | 10 | 1103 ± 119 | NA | NA |
| Compound A | 10 | 1007 ± 127 | 89.6 | .617 |
| (30 mg/kg, QD × 32, PO; day 0-31) | | | | |
| Cisplatin (1.5 mg/kg) + Gemcitabine (40 mg/kg) | 10 | 907 ± 85 | 78.0 | .282 |
| (Q7D × 4, IP; day 5, 12, 19, 26) | | | | |
| Compound A (30 mg/kg, QD × 32, PO; day 0-31) + | 10 | 629 ± 84 | 48.2 | .002* |
| Cisplatin (1.5 mg/kg) + Gemcitabine (40 mg/kg) | | | | |
| (Q7D × 4, IP: day 5, 12, 19, 26) | | | | |

Analysis for Tumor Volume is based on Random Measures ANOVA, Log 10 Volume and Spatial Power covariance structure vs. vehicle. Mean tumor volumes (±SEM) are calculated from the anti-log of the least squares means predicted by the Random Measures ANOVA model on log tumor volume.
Delta T/C % is calculated when the endpoint tumor volume in a treated group is at or above baseline tumor volume. The formula is 100 * (T − Tₒ)/(C − Cₒ), where T and C are endpoint tumor volumes (day 31) in the treated or control group, respectively. Tₒ and Cₒ are baseline (randomization) tumor volumes in those groups (day −1).
*Significant (p < 0.05)
NA: Not Applicable

### EXAMPLE 2

### In Vivo Tumor Growth Inhibition in IDH1 Mutant Cholangiocarcinoma PDX Tumor Model

Compounds and Formulation. For in vivo studies, each test article is prepared at an appropriate concentration with vehicle. Compound A is formulated in Acacia vehicle (water, 10% Acacia, 0.05% Antifoam [Dow Corning 1510-US]) with 1.1 molar equivalents HCl. Compound A is prepared fresh at the appropriate concentrations with vehicle every 7 days and stored at 4° C. between doses. Cisplatin is prepared at 1 mg/ml in 0.9% injectable saline and stored at 4° C. Gemcitabine is prepared at 20 mg/ml in 0.9% injectable saline and stored at −80° C. On the day of administration for both cisplatin and gemcitabine, the appropriate concentration is prepared fresh by dilution in 0.9% injectable saline.

In Vivo Tumor Growth Inhibition Study in IDH1 Mutant (R132C) Intrahepatic Cholangiocarcinoma PDX Tumor Model. Tumor fragments are harvested from host animals and subcutaneously implanted into 6-8 week old female Balb/c nude mice. Mice are fed ad libitum on normal chow. The study is initiated at a mean tumor volume of approximately 146 mm³. Each test article is prepared as described above at the appropriate concentration with vehicle to give animals the doses tested in this study at a dosing volume of 10 μL/gram body weight. Mice are administered Compound A (PO, QD) on day 1 by oral gavage and are treated at the indicated doses (10 mg/kg or 30 mg/kg) for the duration of the study (90 days). On day 5 of Compound A dosing and Q7D×12 thereafter, prepare gemcitabine and cisplatin in appropriate vehicle. Dose gemcitabine (30 mg/kg) and cisplatin (1.5 mg/kg) by intraperitoneal administration. Tumor growth and body weight are monitored over time to evaluate efficacy and signs of toxicity. Bidimensional measurements of tumors are performed twice a week and tumor volumes are calculated based on the following formula: (Tumor Volume)=[(L)×(W2)×0.5] where L is mid-axis length and W is mid-axis width. Mean tumor volumes on day 90 are shown in Table 2.

Compound A, cisplatin plus gemcitabine, and the triple combination of Compound A with cisplatin and gemcitabine are found to have delta T/C % values as provided in Table 2 below. These results indicate that Compound A single agent treatment and in combination with cisplatin and gemcitabine result in statistically significant tumor growth inhibition in a mutant IDH1 (R132C) cholangiocarcinoma patient-derived xenograft model. The addition of Compound A to the cisplatin-gemcitabine regimen demonstrates no evidence of antagonism or overt toxicity, as compared to the cisplatin-gemcitabine treatment alone.

TABLE 2
In vivo tumor growth inhibition in IDH1 mutant cholangiocarcinoma
PDX tumor model implanted in mice

| Group | N | Mean ± SEM | Delta T/C % | n-value |
|---|---|---|---|---|
| Vehicle | 10 | 939 ± 184 | NA | NA |
| Compound A (10 mg/kg, PO, QD × 90) | 10 | 399 ± 122 | 32.8 | .015* |
| Compound A (30 mg/kg, PO, QD × 90) | 10 | 326 ± 102 | 23.7 | .003* |
| Cisplatin (1.5 mg/kg) + Gemcitabine (30 mg/kg) | 10 | 305 ± 74 | 21.0 | .002* |
| (IP, Q7D × 13, starting on day 5) | | | | |
| Compound A (10 mg/kg, PO, QD × 90) + | 10 | 219 ± 44 | 10.4 | <.001* |
| Cisplatin (1.5 mg/kg) + Gemcitabine (30 mg/kg) | | | | |
| (IP, Q7D × 13, starting on day 5) | | | | |
| Compound A (30 mg/kg, PO, QD × 90) + | 10 | 195 ± 49 | 7.3 | <.001* |
| Cisplatin (1.5 mg/kg) + Gemcitabine (30 mg/kg) | | | | |
| (IP, Q7D × 13, starting on day 5) | | | | |

Analysis for Tumor Volume is based on Random Measures ANOVA, Log 10 Volume and Spatial Power covariance structure vs. vehicle. Mean tumor volumes (±SEM) are calculated from the anti-log of the least squares means predicted by the Random Measures ANOVA model on log tumor volume.
Delta T/C % is calculated when the endpoint tumor volume in a treated group is at or above baseline tumor volume. The formula is 100 * (T − Tₒ)/(C − Cₒ), where T and C are endpoint tumor volumes (day 90) in the treated or control group, respectively. Tₒ and Cₒ are baseline (randomization) tumor volumes in those groups (day 0).
*Significant (p < 0.05)
NA: Not Applicable

### EXAMPLE 3

### A Phase 1 Study of Compound a Administered to Patients with Advanced Solid Tumors with IDH1 Mutations

The primary objective of a Phase 1 dose escalation is to determine the maximum tolerated dose (MTD)/recommended Phase 2 dose (RP2D) of Compound A monotherapy when administered to patients with IDH1 R132-mutant advanced solid tumors.

The primary objective of a Phase 1 dose expansion is to assess the preliminary anti-tumor activity of Compound A when administered alone or in combination with cisplatin plus gemcitabine by determining objective response rate (ORR) using Response Evaluation Criteria in Solid Tumors version 1.1 (RECIST 1.1) or Response Assessment in Neuro-Oncology (RANO), as appropriate based on tumor type.

The secondary objectives are (a) to assess the safety and tolerability of Compound A when administered alone or in combination with cisplatin plus gemcitabine; (b) to assess the preliminary anti-tumor activity of Compound A monotherapy and in combination with cisplatin plus gemcitabine based on (i) duration of response (DOR), (ii) time to response (TTR), (iii) progression-free survival (PFS), (iv) disease control rate (DCR), (v) overall survival (OS), (v) changes in serum tumor marker CA 19-9 in patients with cholangiocarcinoma; (c) to characterize the pharmacokinetics (PK) properties of Compound A when administered alone or in combination with cisplatin plus gemcitabine; and (vi) to characterize the pharmacodynamic properties of Compound A as expressed by change in 2-hydroxyglutarate oncometabolite levels in plasma.

Table 3 lists the Compound A monotherapy doses and cycle lengths that are used to determine MTD and R2PD.

TABLE 3
Compound A monotherapy (28 day cycle length)

| Dose Levels | Proposed Doses^{a} | |
|---|---|---|
| DL −1 | 10 | mg QD |
| DL 1 (starting dose) | 25 | mg QD |
| DL 2 | 25 | mg BID |
| DL 3 | 50 | mg BID |
| DL 4 | 100 | mg BID |
| DL 5 | 200 | mg BID |

Abbreviation: BID = twice daily; DL = dose level; PK = pharmacokinetics; QD = daily; SRC = safety review committee.
^{a}lower or intermediate dose levels or dose levels above the currently planned maximum dose of 200 mg BID as well as alternative dosing schedules may be considered by safety review committee upon review of safety, PK and pharmacodynamics; data from previous cohort.

The first dose level was completed using a dose of 25 mg QD. Following review of available PK/PD and safety data, the dosing plan was modified to 50 mg QD as the dose and regimen for DL 2, 100 mg QD for DL 3, 200 mg QD for DL 4, and 400 mg QD for DL 5.

After the MTD or R2PD of Compound A have been determined, a cohort of subjects are evaluated with Compound A in combination with cisplatin plus gemcitabine in patients with IDH1 R132-mutant advanced cholangiocarcinoma and measurable disease who have not received prior therapy for advanced disease (Table 4). For safety lead-in, up to 6 patients are enrolled and treated at the Compound A monotherapy RP2D in combination with cisplatin plus gemcitabine. Safety lead-in patients complete a 21-day DLT evaluation period before additional patients are enrolled into the cohort. The i3+3 decision rules for a cohort size of 6 are used to determine early stopping, with an ‘escalate’ move replaced by a ‘stay’ move. Specifically, if no more than 1 DLTs are observed in the first 6 patients, continued enrollment can be allowed to the cohort. If 2 or more DLTs are observed in the first 6 patients, the Compound A dose can be reduced by 1 level or more, and enrollment continues to the cohort. Once safety of the combination has been confirmed, enrollment continues to a total of approximately 20 patients (including any safety lead-in patients treated at the final expansion dose).

TABLE 4
Compound A combination with cisplatin plus
gemcitabine dosing (21 day cycle length)

| Study Drug | | | Dose Cycle |
|---|---|---|---|
| Administration | Dose Levels | Doses | (days) |
| Compound A | DL 1 | RP2Dm-1 level | C1-n; D1-D21 |
| (oral) | DL 1 (starting | RP2Dm | C1-n; D1-D21 |
| | dose) | | |
| cisplatin (IV) | fixed dose | 25 mg/m² | C1-n^{a}; D1 and D8 |
| gemcitabine (IV) | fixed dose | 1000 mg/m² | C1-n^{a}; D1 and D8 |

Abbreviations: C = cycle; D = day; DL = dose level; n = cycle number; RP2Dm = recommended Phase 2 dose monotherapy; IV = intravenous.
^{a}Planned for a total of 6 to 8 cycles, based on discretion of treating investigator. If the treating investigator feels that treatment beyond 8 cycles is in the patient's best interest, this will be permitted following sponsor approval.

1. A method of treating a solid tumor cancer in a subject having an IDH mutation, comprising administering to the subject a therapeutically effective amount of
(a) a compound of Formula I: 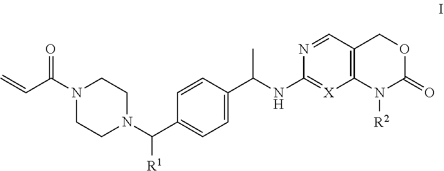
wherein:
R¹ is —CH₂CH(CH₃)₂, —CH₂CH₃, —CH₂CH₂OCH₃, or —CH₂-cyclopropyl;
R² is —CH₃ or —CH₂CH₃; and
X is N or CH,
or a pharmaceutically acceptable salt thereof;
(b) a deoxyadenosine analog, or a pharmaceutically acceptable salt thereof; and
(c) a platinum agent.

2. (canceled)

3. The method of claim 1, wherein the IDH mutation is an IDH1 mutation.

4. The method of claim 3, wherein the IDH1 mutation is an IDH1 R132 mutation.

5. The method of claim 1, wherein the IDH mutation is an IDH2 mutation.

6. The method of claim 5, wherein the IDH2 mutation is an IDH2 R140 or IDH2 R172 mutation.

7. The method of claim 1, wherein X is N, or a pharmaceutically acceptable salt thereof.

8. The method of claim 7, wherein X is N, R¹ is —CH₂-cyclopropyl, and R² is —CH₂CH₃, or a pharmaceutically acceptable salt thereof.

9. The method of claim 1, wherein the compound of Formula I is:
7-[[(1S)-1-[4-[(1R)-2-cyclopropyl-1-(4-prop-2-enoylpiperazin-1-yl)ethyl]phenyl]ethyl]amino]-1-ethyl-4H-pyrimido[4,5-d][1,3]oxazin-2-one;
7-[[(1S)-1-[4-[(1S)-2-cyclopropyl-1-(4-prop-2-enoylpiperazin-1-yl)ethyl]phenyl]ethyl]amino]-1-ethyl-4H-pyrimido[4,5-d][1,3]oxazin-2-one; or
1-Ethyl-7-[[(1S)-1-[4-[1-(4-prop-2-enoylpiperazin-1-yl) propyl]phenyl]ethyl]amino]-4H-pyrimido[4,5-d][1,3]oxazin-2-one;
or a pharmaceutically acceptable salt thereof.

10. The method of claim 1, wherein the compound of Formula I is 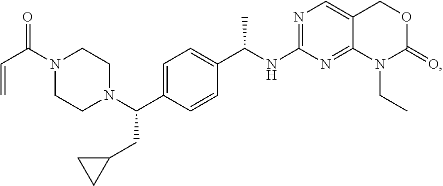 [SMILES: C=CC(=O)N1CCN([C@@H](CC2CC2)c2ccc([C@H](C)Nc3ncc4c(n3)N(CC)C(=O)OC4)cc2)CC1]
or a pharmaceutically acceptable salt thereof.

11. The method of claim 10, wherein the compound of Formula I is

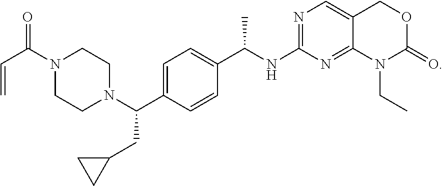 [SMILES: C=CC(=O)N1CCN([C@@H](CC2CC2)c2ccc([C@H](C)Nc3ncc4c(n3)N(CC)C(=O)OC4)cc2)CC1]

12. The method of claim 1, wherein the deoxyadenosine analog is cytarabine, or a pharmaceutically acceptable salt thereof, or gemcitabine, or a pharmaceutically acceptable salt thereof.

13. The method of claim 12, wherein the deoxyadenosine analog is gemcitabine, or a pharmaceutically acceptable salt thereof.

14. The method of claim 13, wherein the deoxyadenosine analog is gemcitabine.

15. The method of claim 1, wherein the platinum agent is cisplatin, carboplatin or oxaliplatin.

16. The method of claim 15, wherein the platinum agent is cisplatin.

17. The method of claim 1, wherein the deoxyadenosine analog is gemcitabine, and the platinum agent is cisplatin.

18. The method of claim 1, wherein the compound of Formula I is: 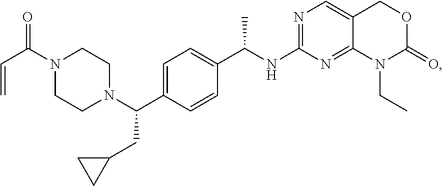 [SMILES: C=CC(=O)N1CCN([C@@H](CC2CC2)c2ccc([C@H](C)Nc3ncc4c(n3)N(CC)C(=O)OC4)cc2)CC1]
the deoxyadenosine analog is gemcitabine, and the platinum agent is cisplatin.

19. The method of claim 1, wherein the solid tumor cancer is cholangiocarcinoma, head and neck cancer, chondrosarcoma, hepatocellular carcinoma, melanoma, pancreatic cancer, astrocytoma, oligodendroglioma, glioma, glioblastoma, bladder carcinoma, colorectal cancer, or lung cancer.

20. The method of claim 19, wherein the solid tumor cancer is cholangiocarcinoma.

21. The method of claim 20, wherein the cholangiocarcinoma is advanced cholangiocarcinoma.

22. The method of claim 1, wherein the solid tumor cancer is cholangiocarcinoma, the compound of Formula I is 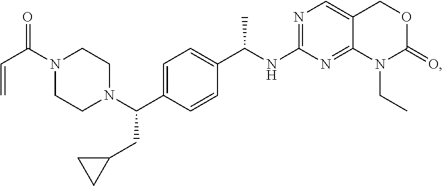 [SMILES: C=CC(=O)N1CCN([C@@H](CC2CC2)c2ccc([C@H](C)Nc3ncc4c(n3)N(CC)C(=O)OC4)cc2)CC1]
the deoxyadenosine analog is gemcitabine, and the platinum agent is cisplatin.

23. The method of claim 22, wherein the cholangiocarcinoma is advanced cholangiocarcinoma.

24.-46. (canceled)
